(19) United States
(12) Patent Application Publication
(10) Pub. No.: US 2003/0050479 A1
(43) Pub. Date: Mar. 13, 2003

(54) Process for the preparation of substituted oxazoles

(76) Inventors: Harald Rust, Neustadt (DE); Kirsten Burkart, Ludwigshafen (DE); Tillmann Faust, Weisenheim (DE); Jochem Henkelmann, Mannheim (DE); Alois Kindler, Grunstadt (DE); Christian Knoll, Neuhofen (DE); Michael Becker, Ludwigshafen (DE)

Correspondence Address: Herbert B. Keil, KEIL & WEINKAUF, 1350 Connecticut Ave., N.W., Washington, DC, 20036, US

(21) Appl. No.: 10/209,529

(22) Filed: Aug. 1, 2002

(30) Foreign Application Priority Data
Aug. 3, 2001 (DE) 10137480.1
Mar. 5, 2002 (DE) 10209446.2

(51) Int. Cl. C07D 263/42

(52) U.S. Cl. 548/228

(57) ABSTRACT

A process for the preparation of 5-alkoxy-substituted oxazoles, in particular for the preparation of 4-methyl-5-alkoxy-substituted oxazoles, and a process for the preparation of pyridoxine derivatives are described.

The present invention relates to a process for the preparation of 5-alkoxy-substituted oxazoles, in particular for the preparation of 4-methyl-5-alkoxy-substituted oxazoles, and to a process for the preparation of pyridoxine derivatives.

5-alkoxy-substituted oxazoles are valuable synthesis units in organic chemistry. 4-methyl-5-alkoxy-substituted oxazoles are of particular significance as important precursors for the synthesis and industrial production of vitamin B₆ (Turchi et al., Chem. Rev. 1975, 75, 416).

A process for the preparation of 5-alkoxy-substituted oxazoles, in particular of 4-methyl-5-alkoxy-substituted oxazoles, which is economical and can be carried out on a large scale is therefore of high importance.

It is known to convert (-isocyanoalkyl acid esters batchwise into the corresponding 5-alkoxy-substituted oxazoles by thermal isomerization.

Itov et al., Khimiko-Farmatsevticheskii Zhurnal, 1978, 12, 102-106 and Mishchenlo et al., Khimiko-Farmatsevticheskii Zhurnal, 1988, 7, 856 to 860 describe a batchwise, thermal cyclization of α-isocyanopropionic acid esters to the corresponding 4-methyl-5-alkoxy-substituted oxazoles at 135° C. The yields of 4-methyl-5-alkoxy-substituted oxazoles achieved by use of various solvents are 4 to 36%. The process has the disadvantage of a low selectivity and thus the disadvantage that large amounts of by-products are formed. The most frequent by-products of this reaction are the unreacted starting material (Yield: 33 to 55%) and the rearranged α-nitrilopropionic acid ester (yield 1 to 39%).

Maeda et al., Bull. Chem. Soc. Japan, 1971, 44, 1407 to 1410 disclose a batchwise, thermal cyclization of various α-isocyanocarboxylic acid esters to the corresponding 5-alkoxy-substituted oxazoles at temperatures from 150 to 180° C. Depending on substituents, yields from 5.1 to 28.2% are achieved.

In JP 54-20493, a batchwise process for the preparation of 4-methyl-5-alkoxy-substituted oxazoles by thermal cyclization of α-isocyanopropionic acid esters at temperatures from 155 to 170° C. in the presence of a tertiary amine is described. After completion of the reaction, the solution is fractionally distilled under reduced pressure at temperatures which are as low as possible. Although improved selectivities of the desired oxazoles are achieved (34 to 91.5%), the low conversion (11.1 to 49.4%) still does not lead to satisfactory yields.

All processes of the prior art have the disadvantage of low conversions and low selectivities and thus low yields of 5-alkoxy-substituted oxazoles.

It is an object of the of the present invention to make available a further process for the preparation of 5-alkoxy-substituted oxazoles having advantageous properties, which no longer has the disadvantages of the prior art and which yields 5-alkoxy-substituted oxazoles in high selectivities and yields with high conversions.

We have found that this object can be achieved by a process for the preparation of 5-alkoxy-substituted oxazoles of the formula I,

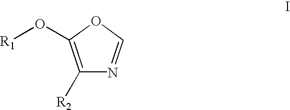

where

R₁ is an optionally substituted C₁-C₆-alkyl radical and

R₂ is hydrogen or an optionally substituted C₁-C₆-alkyl radical,

by converting α-isocyanoalkyl acid esters of the formula II

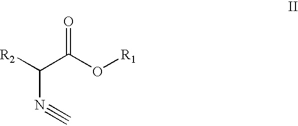

in the presence of bases

at temperatures of greater than 80° C.

into the 5-alkoxy-substituted oxazoles of the formula I

and, simultaneously to the conversion, separating the 5-alkoxy-substituted oxazoles of the formula I from the reaction mixture.

An optionally substituted C₁-C₆ alkyl radical is understood as meaning for the radicals R₁ and R₂, independently of one another, branched or unbranched, optionally substituted C₁-C₆-alkyl radicals, such as, for example, optionally substituted methyl, ethyl, propyl, 1-methylethyl, n-butyl, 1-methylpropyl, 2-methylpropyl, 1,1-dimethylethyl, pentyl, 1-methylbutyl, 2-methylbutyl, 1,2-dimethylpropyl, 1,1-dimethylpropyl, 2,2-dimethylpropyl, 1-ethylpropyl, hexyl, 1-methylpentyl, 1,2-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl.

The nature of the substituents is not critical. The C₁-C₆-alkyl radicals can contain up to 6 substituents, depending on the free bond possibilities, preferably selected from the group consisting of aryl, hydroxyaryl, —NO₂, —NH₂, —OH, —CN, —COOH, or halogen, in particular F or Cl.

In a preferred embodiment, the C₁-C₆-alkyl radicals of the radicals R₁ and R₂ are not substituted.

Preferred radicals for R₁ are C₁-C₄-alkyl radicals, such as, for example, methyl, ethyl, isopropyl, n-propyl, n-butyl, sec-butyl or tert-butyl, particular preferably n-butyl.

Preferred radicals for R₂ are hydrogen and C₁-C₄-alkyl radicals, such as, for example, methyl, ethyl, iso-propyl, n-propyl, n-butyl, sec-butyl or tert-butyl, particularly preferably methyl.

The combination of the preferred radicals for R₁ and R₂ is preferred; the combination R₁=n-butyl and R₂=methyl is particularly preferred.

In a particularly preferred embodiment of the process according to the invention, n-butyl α-isocyanopropionate is accordingly converted into 4-methyl-5-n-butoxyoxazole.

The α-isocyanoalkyl acid esters of the formula II used in the process according to the invention can be employed in any desired purity.

The α-isocyanoalkyl acid esters of the formula II can be prepared in a manner known per se from the corresponding formamido acid esters of the formula V

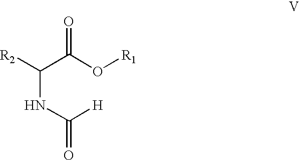

by reaction with phosphorus oxychloride or phosgene in the presence of bases. Customary synthesis methods are described in Itov et al., Khimiko-Farmatsevticheskii Zhurnal, 1978, 12, 102-106; Maeda et al., Bull. Chem. Soc. Japan, 1971, 44, 1407-1410; Ugi et al., Chem. Ber. 1961, 94, 2814; Chem. Ber. 1960, 93, 239-248, Angew. Chem. 1965, 77, 492-504, Chem. Ber. 1975, 1580-1590, DE 30 29 231 A1 and J. Heterocyclic Chemistry 1988, 17, 705.

Bases in the process according to the invention are understood as meaning compounds having Brönsted base properties. Preferred bases are tertiary amines, such as, for example, triethylamine, triisopropylamine, tri-n-butylamine, dimethylcyclohexylamine, tris(2-ethylhexyl)amine, N-methylpyrrolidone, N,N,N′N′-tetramethyl-1,3-propanediamine, N,N-diethylaniline or N,N-dibutylaniline. The use of tri-n-butylamine as a base is particularly preferred.

Below 80° C., no noticeable thermal cyclization takes place. The temperature in the conversion according to the invention is therefore at least 80° C.

In a preferred embodiment, the process according to the invention takes place at temperatures from 100 to 200° C., particularly preferably at temperatures from 120 to 170° C., very particularly preferably at temperatures from 130 to 170° C.

The molar ratio of base to (-isocyanoalkyl acid ester of the formula II is not critical and is preferably 10:1 to 0.05:1.

The process according to the invention can be carried out, in particular, batchwise, semi-batchwise or continuously.

In a preferred embodiment of the process according to the invention, the process is carried out batchwise or semi-batchwise.

Batchwise is understood as meaning a batchwise procedure. In this, the α-isocyanoalkyl acid esters of the formula II and the bases are preferably introduced into a reactor and the α-isocyanoalkyl acid esters are converted into the 5-alkoxy-substituted oxazoles of the formula I at temperatures greater than 80° C., the 5-alkoxy-substituted oxazoles of the formula I being separated from the reaction mixture simultaneously to the conversion.

There are many embodiments of reactors which are suitable for the preferred batchwise procedure. Preferred reactors should have the property of making a conversion possible with simultaneous separation of a reaction product.

For example, back-mixed reactors, such as, for example, loop reactors and customary batchwise reactors, such as, for example, vessels in all embodiments, having an attached reaction column can be used as reactors for the batchwise procedure.

Semi-batchwise is understood as meaning a semi-batchwise procedure. In this, the u-isocyanoalkyl acid esters of the formula II and the bases are preferably fed to a reactor semi-continuously and the (-isocyanoalkyl acid esters are converted into the 5-alkoxy-substituted oxazoles of the formula I at temperatures greater than 80° C., the 5-alkoxy-substituted oxazoles of the formula I being separated from the reaction mixture simultaneously to the conversion.

There are many embodiments of the reactors which are suitable for the semi-batchwise procedure. Preferred reactors should have the property of making a conversion possible with simultaneous separation of a reaction product.

For example, reactors which can be used for the semi-batchwise procedure are loop reactors, membrane reactors and customary semi-batchwise reactors, such as vessels in all embodiments, having an attached reaction column.

In a preferred embodiment, the process is carried out in a batchwise or semi-batchwise reactor having an attached reaction column and, simultaneously to the conversion, the 5-alkoxy-substituted oxazoles of the formula I are separated from the reaction mixture by rectification.

As the person skilled in the art knows, under the term “column” below, if not mentioned otherwise, a column construction having a bottom is understood.

An “attached column” is correspondingly understood as meaning only the column construction without a bottom.

Reaction columns or attached reaction columns are preferably understood as meanings columns whose fittings have a hold-up, i.e., for example, columns containing plates, fillings, packings or filling materials.

Particularly advantageous column plates make a high residence time of the liquid possible, the residence time on the fittings of the reaction column preferably being at least 30 min.

Preferred column plates are, for example, valve plates, preferably bubble-cap plates or related types such as, for example, tunnel plates, Lord stages and other fittings or Thormann plates.

Preferred structured packings are, for example, structured packings of the type Mellapack® (Sulzer), BY® (Sulzer), B1® (Montz) or A3® (Montz) or packings with comparable embodiments.

The reaction columns or attached reaction columns can be designed in any desired manner depending on the type and the fittings. The use of a dividing wall column as a reaction column is particularly preferred.

A reaction column or attached reaction column which can be designed in very different types has the property, as a reactor, of simultaneously making possible a conversion of reactants and the separation of the 5-alkoxy-substituted oxazoles of the formula I from the reaction mixture by rectification.

In this preferred embodiment of the batchwise or semi-batchwise procedure using a batchwise or semi-batchwise reactor having an attached reaction column, it is furthermore advantageous to adjust the rectification parameters such that

D the conversion of the α-isocyanoalkyl acid esters of the formula II into the 5-alkoxy-substituted oxazoles of the formula I takes place in the reactor and/or on the fittings of the attached reaction column and

E the 5-alkoxy-substituted oxazoles of the formula I formed during the conversion are separated by means of the attached reaction column.

Depending on the form of design of the reactor, the attached reaction column and the reactants used, this is achieved by different adjustments of the rectification parameters. Suitable rectification parameters are, for example, temperature, pressure, reflux ratio in the column, design of the column and its fittings, heat conduction or energy input, which the person skilled in the art can optimize by means of routine tests such that the features D and E are achieved.

In the batchwise and semi-batchwise procedure, the pressure at the column head of the attached reaction column is adjusted such that the temperature in the reactor and on the fittings is at least 80° C., preferably 100 to 200° C., particularly preferably 120 to 170° C., very particularly preferably 130 to 170° C.

Typically, the head pressure of the column is adjusted to 5 to 800 mbar, such that the bottom pressure resulting therefrom, depending on the type of column used and, if appropriate, the type of column fittings used, is typically 5 mbar to atmospheric pressure.

It is possible that the 5-alkoxy-substituted oxazoles of the formula I form an azeotropic mixture with the bases used, such that the 5-alkoxy-substituted oxazoles of the formula I are separated as an azeotropic mixture by means of the attached column.

In this case, it is advantageous to adjust the head pressure and therewith automatically also the bottom pressure in the column, depending on the 5-alkoxy-substituted oxazole of the formula I prepared and the base used, such that the proportion of base in the azeotrope in the head flow is as low as possible.

The separation of the base from the azeotrope is in this case carried out in a manner known per se, for example by means of a subsequent second rectification using a different pressure (two-pressure distillation).

For example, the 4-methyl-5-n-butoxyoxazole prepared by the process according to the invention forms an azeotrope with the base tri-n-butylamine. On setting a head pressure of 100 mbar, the azeotrope in the head flow is composed of 91% by weight of 4-methyl-5-n-butoxyoxazole and 9% by weight of tri-n-butylamine.

The separation of tri-n-butylamine from the azeotrope can in this case be carried out, for example, by a subsequent second rectification at a head pressure of 10 mbar.

The batchwise and semi-batchwise procedure can be carried out in the presence or absence of solvents. In a preferred embodiment, the process is carried out without solvent.

However, it is possible to add solvents or to employ crude mixtures in the process according to the invention which, in addition to the Q-isocyanoalkyl acid esters of the formula II and the bases, already contain solvent.

In a further preferred embodiment, the process according to the invention is carried out in the presence of an inert solvent. An inert solvent is preferably understood as meaning nonpolar and polar aprotic solvents such as toluene, xylene or chlorobenzene, dichloromethane, dichloroethane, dichlorobenzene, ethylene carbonate, propylene carbonate, in particular chlorobenzene.

In this case, in the batchwise or semi-batchwise procedure firstly the more easily boiling solvent and subsequently the 5-alkoxy-substituted oxazole of the formula I are separated by rectification.

In a particularly preferred embodiment of the process according to the invention, the procedure is carried out continuously.

In the continuous procedure, the α-isocyanoalkyl acid esters of the formula II and the bases are fed continuously to a reactor either as a mixture or separately, the α-isocyanoalkyl acid esters of the formula II are converted into the 5-alkoxy-substituted oxazoles of the formula I in the reactor and subsequently the reaction products are continuously removed from the reactor, the 5-alkoxy-substituted oxazoles of the formula I being separated continuously from the reaction mixture simultaneously to the conversion.

There are many designs of reactors which are suitable for the particularly preferred continuous procedure. Preferred reactors should have the property of making possible a continuous conversion with simultaneous separation of a reaction product.

For example, reactors which can be used for the continuous procedure are stills having an attached column, extraction columns, bubble-cap plate columns, membrane reactors, Lord reactors or reaction columns.

As mentioned above, the term column, if not mentioned otherwise, is understood as meaning a column construction having a bottom.

Reaction columns are preferably understood as meaning columns whose fittings have a hold-up, i.e., for example, columns having plates, fillings, packings or filling materials.

In a particularly preferred embodiment of the process according to the invention, the continuous procedure is carried out in a reaction column as a reactor, the 5-alkoxy-substituted oxazoles of the formula I being separated continuously from the reaction mixture by rectification simultaneously to the conversion.

The reaction columns can be designed in any desired manner as regards the type of construction and the fittings. The use of a dividing wall column as a reaction column is particularly preferred.

A reaction column which can be designed in very different types has the property as a reactor of simultaneously making possible a conversion of reactants and the separation of the 5-alkoxy-substituted oxazoles of the formula I from the reaction mixture by rectification.

In this particularly preferred embodiment using a reaction column for the continuous procedure, it is furthermore advantageous to adjust the rectification parameters such that

A the conversion of the (-isocyanoalkyl acid esters of the formula II into the 5-alkoxy-substituted oxazoles of the formula I takes place on the fittings and, if appropriate, in the bottom of the reaction column,

B the 5-alkoxy-substituted oxazoles of the formula I formed in the conversion are separated continuously with the head stream or side stream of the reaction column and

C the base, and also the high-boiling components which may be formed in the conversion are separated continuously and independently of one another with the bottom stream or side stream of the reaction column.

Depending on the form of design of the reaction column and the reactants used, this is achieved by means of different adjustments of the rectification parameters. Suitable rectification parameters are, for example, temperature, pressure, reflux ratio in the column, design of the column and its fittings, heat conduction and residence time, in particular in the bottom, or energy input, which the person skilled in the art can optimize by means of routine tests such that the features A, B and C are achieved.

In feature C, the base can in particular be separated separately from the high-boiling components in a second side stream. Side stream is understood according to the invention as meaning the continuous discharge of a substance via a side outlet of the column.

For the continuous procedure of the process according to the invention also, the pressure at the column head is adjusted such that the temperature in the bottom and at the fittings is at least 80° C., preferably 100 to 200° C., particularly preferably 120 to 170° C., very particularly preferably 130 to 170° C.

Typically, the head pressure of the column is adjusted to 5 to 800 mbar for the continuous procedure such that the bottom pressure resulting therefrom, depending on the type of column used and, if appropriate, the types of column fitting used, is typically 5 mbar to atmospheric pressure.

The residence time in the reaction column for the continuous procedure is typically between 10 minutes and 7 hours, preferably between 30 minutes and 4 hours.

It is also possible in the continuous procedure that the 5-alkoxy-substituted oxazoles of the formula I form an azeotropic mixture with the bases used, such that the 5-alkoxy-substituted oxazoles of the formula I are separated as an azeotropic mixture via the head stream.

In this case it is advantageous to adjust the head pressure and therewith automatically also the bottom pressure in the column, depending on the 5-alkoxy-substituted oxazole of the formula I prepared and the base used, such that the proportion of base in the azeotrope in the head stream is as low as possible.

The separation of the base from the head stream azeotrope is in this case carried out in a manner known per se, for example by a subsequent second rectification using a different pressure (two-pressure distillation).

The continuous procedure of the process according to the invention can be carried out in the presence or absence of solvents. In a preferred embodiment, the continuous procedure of the process according to the invention is carried out without solvent.

In a further preferred embodiment, the continuous procedure of the process according to the invention is carried out in the presence of an inert solvent. An inert solvent is preferably understood as meaning nonpolar and polar aprotic solvents such as toluene, xylene or chlorobenzene, dichloromethane, dichloroethane, dichlorobenzene, ethylene carbonate, propylene carbonate, in particular chlorobenzene.

In the case of the use of a solvent, the solvent, for example with the base and the (-isocyanoalkyl acid esters of the formula II as a mixture, or each individual component separately can be fed continuously to the column.

In the case of the use of an inert solvent in the continuous procedure of the process according to the invention, the rectification parameters are preferably adjusted such that

A the conversion of the (-isocyanoalkyl acid esters of the formula II into the 5-alkoxy-substituted oxazoles of the formula I takes place on the fittings and, if appropriate, in the bottom of the reaction column,

B1 in the case where the solvent has a higher boiling point than the 5-alkoxy-substituted oxazoles of the formula I formed in the conversion, the 5-alkoxy-substituted oxazoles of the formula I are separated continuously with the head stream and the solvent is separated continuously via the side stream or bottom stream of the reaction column,

B2 in the case where the solvent has a lower boiling point than the 5-alkoxy-substituted oxazoles of the formula I formed in the conversion, the 5-alkoxy-substituted oxazoles of the formula I are separated continuously with a side stream and the solvent is separated continuously with the head stream of the reaction column and

C the base, and also the high-boiling components which may be formed in the conversion are separated continuously and independently of one another with the bottom stream or side stream of the reaction column.

Fittings of the reaction column which can be used are any embodiments, such as, for example, column plates, fillings, filling materials or structured packings.

Particularly advantageous column plates make possible a high residence time of the liquid, the residence time on the fittings of the reaction column preferably being at least 30 min.

Preferred column plates are, for example, valve plates, preferably bubble-cap plates or related types of construction such as, for example, tunnel plates, Lord stages and other fittings or Thormann plates.

Preferred structured packings are, for example, structured packings of the type Mellapack® (Sulzer), BY® (Sulzer), B1® (Montz) or A3® (Montz) or packings with comparable embodiments.

The process according to the invention has the following advantages compared with the prior art:

Using the process according to the invention, selectivities of over 95% based on the (-isocyanoalkyl acid esters of the formula II employed are achieved.

The conversion is almost 100%, so that the yields of 5-alkoxy-substituted oxazoles of the formula I are over 95% based on the α-isocyanoalkyl acid esters of the formula II employed.

The particularly preferred continuous procedure has the further advantage of a markedly greater space-time yield than in the processes known hitherto.

The process according to the invention is a novel and advantageous partial synthesis step in the process for the preparation of pyridoxine derivatives of the formula IX,

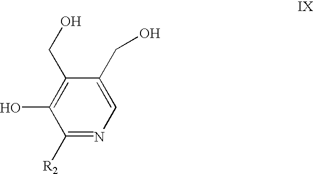

in particular for the preparation of pyridoxine (vitamin B₆; 10 formula IX, R₂=methyl).

The invention therefore furthermore relates to a process for the preparation of pyridoxine derivatives of the formula 1×

in which amino acids of the formula III

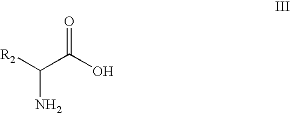

are converted into amino acid esters of the formula IV,

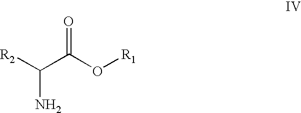

these are converted into formamido acid esters of the formula V

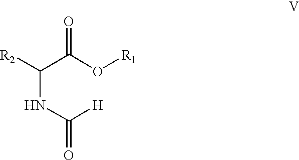

these are converted into (-isocyanoalkyl acid esters of the formula II,

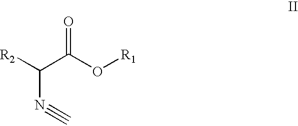

these are converted in the presence of bases

at temperatures of greater than 80° C.

into the 5-alkoxy-substituted oxazoles of the formula I

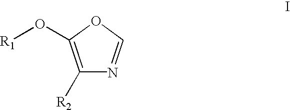

and, simultaneously to the conversion, the 5-alkoxy-substituted oxazoles of the formula I are separated from the reaction mixture,

the 5-alkoxy-substituted oxazoles of the formula I are reacted with protected diols of the formula VI,

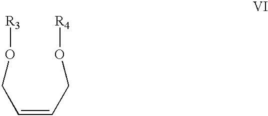

where

R₃, R₄ independently of one another or R₃ and R₄ together are a protective group of the hydroxyl function,

to give the Diels-Alder adducts of the formula VII,

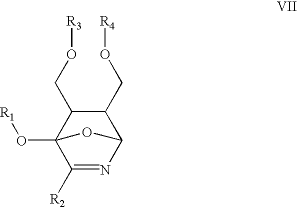

and these are converted into the pyridoxine derivatives of the formula IX by acid treatment and removal of the protective group.

The entire process is known, except for the novel, advantageous substep of the conversion of a-isocyanoalkyl acid esters of the formula II into 5-alkoxy-substituted oxazoles of the formula I, from Ullmann's Encyclopedia of Industrial Chemistry 1996, Vol. A 27, pages 533 to 537.

Starting substances for the entire synthesis are inexpensive amino acids of the formula III, preferably alanine (R₂=methyl). These are converted into amino acid esters of the formula IV in a manner known per se, for example by acid-catalyzed esterification with the alcohols R₁—OH, preferably n-butanol. This esterification, however, can also be achieved by other methods, such as, for example, by activation of the acid function and base-catalyzed esterification. Further methods are described in U.S. Pat. No. 3,227,721.

The amino acid esters of the formula IV are converted into the formamido acid esters of the formula V in a manner known per se, for example as described in U.S. Pat. No. 3,227,721.

The formamido acid esters of the formula V are subsequently converted into the α-isocyanoalkyl acid esters of the formula II in a manner known per se, as described above.

The α-isocyanoalkyl acid esters of the formula II are converted into the 5-alkoxy-substituted oxazoles of the formula I using the process according to the invention, as described above.

In the preferred overall process, this substep is carried out as described above in the preferred embodiments.

The 5-alkoxy-substituted oxazoles of the formula I are subsequently reacted with protected diols of the formula VI to give the Diels-Alder adducts of the formula VII.

This substep can be connected to the process according to the invention at a later stage, but in the continuous procedure of the process according to the invention it can also be carried out by continuous supply of the protected diols of the formula VI to the reactor of the process according to the invention simultaneously to the conversion of the (-isocyanoalkyl acid esters of the formula II into the 5-alkoxy-substituted oxazoles of the formula I. The supply is carried out here either as a mixture with the α-isocyanoalkyl acid esters of the formula II, the base and, if appropriate, the solvent or as a separate component. As a product, the 5-alkoxy-substituted oxazoles are in this case removed directly via the bottom discharge of the column in the form of their Diels-Alder adduct.

The radicals R₃, R₄ are independently of one another understood as meaning a protective group, preferably an acid-labile protective group of the hydroxyl function.

In principle, any acid-labile protective group can be used. Preferred acid-labile protective groups are the acid-labile protective groups for hydroxyl groups known in the literature (T. W. Greene, Protective Groups in Organic Synthesis, John Wiley & Sons New York, 1981, pages 14-71; P. J. Kocienski, Protecting Groups, Georg Thieme Verlag Stuttgart, 1994, pages 21-94).

Furthermore, in a preferred embodiment the radicals R₃ and R₄ can together form an acid-labile protective group of the two hydroxyl functions. Preferably, to this end the two hydroxyl functions form a cyclic acetal with ketones or aldehydes, such as, for example, acetone or isobutyraldehyde.

By means of subsequent acid treatment of the Diels-Alder adducts of the formula VII, aromatization to the pyridoxine structure takes place with elimination of the alcohol R₁-OH. The removal of the acid-labile protective group(s), which as a rule is carried out by means of acidic, aqueous treatment, yields the pyridoxine derivatives of the formula IX, in particular pyridoxine (vitamin B6, R₂=methyl).

The alcohol R₁—OH and the protective groups R₃ and R₄ can be recovered and employed again in the overall process.

The use of the novel advantageous substep according to the invention in the overall process leads to an increase in the total yield.

The examples below illustrate the invention.

### EXAMPLE 1

Continuous Preparation of 4-methyl-5-n-butoxyoxazole in a Dividing Wall Column

A mixture of 20.5% by weight of n-butyl U-isocyanopropionate (R₁=n-butyl, R₂=methyl) and 79.5% by weight of tri-n-butylamine was introduced into a continuously operated dividing wall column (4.8 m×64 mm) packed with 3×3 mm V₂A-Raschig rings and a partition of height 2.4 m having 60 theoretical separating stages.

4-Methyl-5-n-butoxyoxazole as an azeotrope with tri-n-butylamine (90:10% by weight) having a boiling point of 158° C. passes over the head at a 500 mbar head pressure and a bottom temperature of 165° C. High-boiling components and tributylamine are drawn off in the column bottom. The conversion was 98.4%, the selectivity 99%. The yield of 4-methyl-5-n-butoxyoxazole was 95% based on the n-butyl (-isocyanopropionate employed.

The azeotrope was subsequently separated in the same column at a head pressure of 10 mbar. As head product, an azeotrope having the composition 4-methyl-5-n-butoxyoxazole:tri-n-butylamine=70:30 is obtained and, in the side outlet, pure 4-methyl-5-n-butoxyoxazole having a boiling point of 98° C. The distillation yield was 99% (40% of pure 4-methyl-5-n-butoxyoxazole and 60% of 4-methyl-5-n-butoxyoxazole as an azeotrope, which was fed back into the first distillation). The pure 4-methyl-5-n-butoxyoxazole had a concentration of 99.8%.

### EXAMPLE 2

Continuous Preparation of 4-methyl-5-n-butoxyoxazole in a Dividing Wall Column with Solvent

A mixture of 13.1% by weight of n-butyl cc-isocyanopropionate (R₁=n-butyl, R₂=methyl), 32,2% by weight of monochlorobenzene and 50.1% by weight of tri-n-butylamine was introduced into a continuously operated dividing wall column (4.8 m×64 mm) packed with 3×3 mm V₂A-Raschig rings and a partition of height 2.4 m having 60 theoretical separating stages.

Monochlorobenzene having a boiling point of 90° C. passes over the head at a 300 mbar head pressure and a bottom temperature of 169° C., 4-methyl-5-n-butoxyoxazole as an azeotrope with tri-n-butylamine (88:12% by weight) is obtained in the side outlet with a passing-over temperature of 151° C. High-boiling components and tributylamine are drawn off in the column bottom. The conversion was 99.5%, the selectivity 99%. The yield of 4-methyl-5-n-butoxyoxazole was 94% based on the n-butyl α-isocyanopropionate employed.

The azeotrope was separated analogously to Example 1.

### EXAMPLE 3

Continuous Preparation of 4-methyl-5-n-butoxyoxazole in a Reaction Column with Solvent

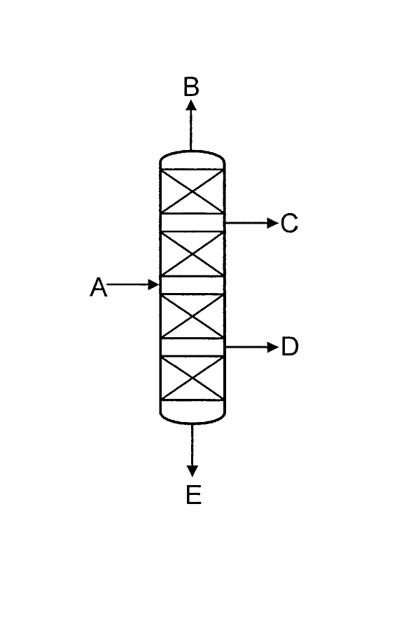

A mixture of 20.6% by weight of chlorobenzene, 5.2% by weight of n-butyl α-isocyanopropionate (R₁=n-butyl, R₂=methyl) and 72.60% by weight of tris(2-ethylhexyl)amine was continuously fed via the intake (A) to a column according to Example 1 but without a partition (see FIG. 1).

The solvent passes through head outlet (B) at a head pressure of 300 mbar and a bottom temperature of 165° C. The 4-methyl-5-n-butoxyoxazole is obtained in a yield of 99% via the side outlet (C). The amine is evacuated via the bottom discharge (E).

### EXAMPLE 4

Continuous Preparation of 4-methyl-5-n-butoxyoxazole in a Reaction Column

According to Example 3, a mixture of 13.14% of n-butyl α-isocyanopropionate and 86.86% of tris(2-ethylhexyl)amine is continuously fed in via the intake (A).

The 4-methyl-5-n-butoxyoxazole is discharged via the head outlet (B) at a head pressure of 400 mbar and a bottom temperature of 165° C. and the amine is evacuated via the bottom discharge (E). The yield of 4-methyl-5-n-butoxyoxazole is 98.8%.

### EXAMPLE 5

Continuous Preparation of 4-methyl-5-isobutoxyoxazole in a Reaction Column

According to Example 3, a mixture of 22.7% of isobutyl α-isocyanopropionate and 77.3% of N,N-dibutylaniline is continuously fed in via the intake (A).

The 4-methyl-5-isobutoxyoxazole is discharged at a temperature of 150° C. via the head outlet (B) at a head pressure of 300 mbar and a bottom temperature of 160° C. The amine is obtained at 161° C. via the side outlet D. The yield of 4-methyl-5-isobutoxyoxazole is 91%.

### EXAMPLE 6

Continuous Preparation of 4-methyl-5-n-butoxyoxazole in a Reaction Column

According to Example 5, a mixture of 11.8% of n-butyl α-isocyanopropionate and 88.2% of N,N-dibutylaniline is continuously fed in via the intake (A).

4-Methyl-5-n-butoxyoxazole is obtained with a yield of 98.7% via the head outlet B and the amine is obtained via the side outlet D.

We claim:

1. A process for the preparation of 5-alkoxy-substituted oxazoles of the formula I, 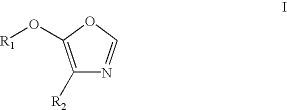
where
R₁ is an optionally substituted C₁-C₆-alkyl radical and
R₂ is hydrogen or an optionally substituted C₁-C₆-alkyl radical,
by converting α-isocyanoalkyl acid esters of the formula II 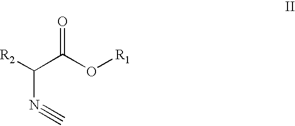
in the presence of bases
at temperatures of greater than 80° C.
into the 5-alkoxy-substituted oxazoles of the formula I
and, simultaneously to the conversion, separating the 5-alkoxy-substituted oxazoles of the formula I from the reaction mixture.

2. A process as claimed in claim 1, wherein the process is carried out batchwise.

3. A process as claimed in claim 2, wherein the process is carried out in a batchwise or semi-batchwise reactor having an attached reaction column and, simultaneously to the conversion, the 5-alkoxy-substituted oxazoles of the formula I are separated from the reaction mixture by rectification.

4. A process as claimed in claim 3, wherein the rectification parameters are adjusted such that
D the conversion of the (X-isocyanoalkyl acid esters of the formula II into the 5-alkoxy-substituted oxazoles of the formula I takes place in the reactor and/or on the fittings of the attached reaction column and
E the 5-alkoxy-substituted oxazoles of the formula I formed during the conversion are separated by means of the attached reaction column.

5. A process as claimed in any of claims 2 to 4, wherein the conversion is carried out in the presence of an inert solvent.

6. A process as claimed in any of claims 3 to 5, wherein the reaction column used is a dividing wall column.

7. A process as claimed in any of claims 4 to 7, wherein the head pressure of the column is adjusted to 5 to 800 mbar and the bottom pressure resulting therefrom, depending on the type of column used and, if appropriate, the type of column fitting used, is 10 mbar to atmospheric pressure.

8. A process as claimed in claim 1, wherein the process is carried out continuously.

9. A process as claimed in claim 8, wherein the process is carried out in a reaction column and, simultaneously to the conversion, the 5-alkoxy-substituted oxazoles of the formula I are separated from the reaction mixture by rectification.

10. A process as claimed in claim 9, wherein the rectification parameters are adjusted such that
A the conversion of the α-isocyanoalkyl acid esters of the formula II into the 5-alkoxy-substituted oxazoles of the formula I takes place on the fittings and, if appropriate, in the bottom of the reaction column,
B the 5-alkoxy-substituted oxazoles of the formula I formed in the conversion are separated continuously with the head stream or side stream of the reaction column and
C the base, and also the high-boiling components which may be formed in the conversion are separated continuously and independently of one another with the bottom stream or side stream of the reaction column.

11. A process as claimed in any of claims 8 to 10, wherein the conversion is carried out in the presence of an inert solvent and the reaction parameters are adjusted such that
A the conversion of the α-isocyanoalkyl acid esters of the formula II into the 5-alkoxy-substituted oxazoles of the formula I takes place on the fittings and, if appropriate, in the bottom of the reaction column,
B1 in the case where the solvent has a higher boiling point than the 5-alkoxy-substituted oxazoles of the formula I formed in the conversion, the 5-alkoxy-substituted oxazoles of the formula I are separated continuously with the head stream and the solvent is separated continuously via the side stream or bottom stream of the reaction column,
B2 in the case where the solvent has a lower boiling point than the 5-alkoxy-substituted oxazoles of the formula I formed in the conversion, the 5-alkoxy-substituted oxazoles of the formula I are separated continuously with a side stream and the solvent is separated continuously with the head stream of the reaction column and
C the base, and also the high-boiling components which may be formed in the conversion are separated continuously and independently of one another with the bottom stream or side stream of the reaction column.

12. A process as claimed in any of claims 9 to 11, wherein the reaction column used is a dividing wall column.

13. A process as claimed in any of claims 9 to 12, wherein, in the case where the base forms an azeotrope with the 5-alkoxy-substituted oxazoles of the formula I, the head pressure in the column is adjusted such that the proportion of base in the azeotrope in the head stream is as low as possible.

14. A process as claimed in any of claims 9 to 13, wherein the head pressure of the column is adjusted to 5 to 800 mbar and the bottom pressure resulting therefrom, depending on the type of column used and, if appropriate, the type of column fitting used, is 10 mbar to atmospheric pressure.

15. A process for the preparation of pyridoxine derivatives of the formula IX 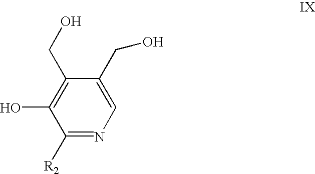
where
R₂ is hydrogen or an optionally substituted C₁-C₆-alkyl radical,
in which amino acids of the formula III 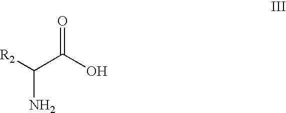
are converted into amino acid esters of the formula IV, 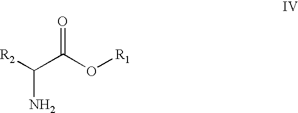
where
R₁ is an optionally substituted C₁-C₆-alkyl radical,
these are converted into formamido acid esters of the formula V 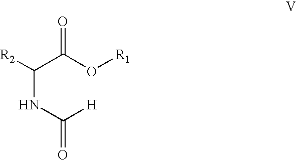
these are converted into (-isocyanoalkyl acid esters of the formula II, 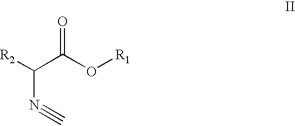
these are converted in the presence of bases
at temperatures of greater than 80° C.
into the 5-alkoxy-substituted oxazoles of the formula I 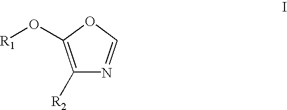
and, simultaneously to the conversion, the 5-alkoxy-substituted oxazoles of the formula I are separated from the reaction mixture,
the 5-alkoxy-substituted oxazoles of the formula I are reacted with protected diols of the formula VI, 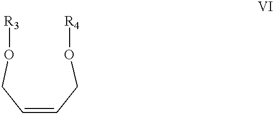
where
R₃, R₄ independently of one another or R₃ and R₄ together are a protective group of the hydroxyl function,
to give the Diels-Alder adducts of the formula VII, 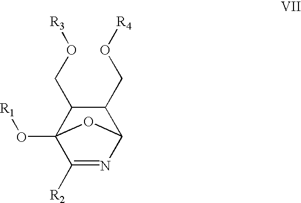
and these are converted into the pyridoxine derivatives of the formula IX by acid treatment and removal of the protective group.
